SEQUENCE LISTING (10 sequences)

SEQ ID NO: 1 - DNA, 23 bp, artificial
  synthetic primer
ggaccgagagactctggaaaatc

SEQ ID NO: 2 - DNA, 24 bp, Artificial
  synthetic primer
gggtcatccttatcttcaactgtc

SEQ ID NO: 3 - DNA, 21 bp, Artificial
  synthetic primer
cggaggccgatccaggtcatg

SEQ ID NO: 4 - DNA, 21 bp, artificial
  synthetic primer
caatcggggatgtctgaggga

SEQ ID NO: 5 - DNA, 24 bp, artificial
  synthetic primer
aagcctgcaaaaataaaaatgatg

SEQ ID NO: 6 - DNA, 24 bp, artificial
  synthetic primer
tgtaaatggtcttgctcttggtct

SEQ ID NO: 7 - DNA, 21 bp, artificial
  synthetic primer
ggccgcccgacaccccaatct

SEQ ID NO: 8 - DNA, 21 bp, artificial
  synthetic primer
atagtgacccgtcccatctcg

SEQ ID NO: 9 - DNA, 20 bp, artificial
  synthetic primer
gaagatggtgatgggatttc

SEQ ID NO: 10 - DNA, 18 bp, artificial
  synthetic primer
gaaggtgaaggtcggagt

(19) United States
(12) Patent Application Publication
(10) Pub. No.: US 2013/0102556 A1
(43) Pub. Date: Apr. 25, 2013

(54) Use of N1,N4-bis[3-(Ethylamino)Propyl]-2-Butene-1,4-Diamine Compounds in Combination with Epigenetic-Acting Pharmaceuticals for Enhanced Cancer Therapy

(71) Applicants: Laurence Marton, Palo Alto, CA (US); Robert A. Casero, Glen Arm, MD (US)

(72) Inventors: Laurence Marton, Palo Alto, CA (US); Robert A. Casero, Glen Arm, MD (US)

(73) Assignee: THE JOHNS HOPKINS UNIVERSITY, Baltimore, MD (US)

(21) Appl. No.: 13/576,692

(22) PCT Filed: Feb. 2, 2011
(86) PCT No.: PCT/US2011/023504

§ 371 Date: Nov. 30, 2012

Related U.S. Application Data
(60) Provisional application No. 61/300,698, filed on Feb. 2, 2010.

(51) Int. Cl. A61K 31/132; A61K 31/4406; A61K 31/706

(52) U.S. Cl. CPC A61K 31/132; A61K 31/706; A61K 31/4406
USPC 514/43; 514/671; 514/357

(57) ABSTRACT

Combination methods for treatment of cancer and of blood disorders, using PG-11047 ((2Z)-N1,N4-bis[3-(ethy-lammo) propyl]-2-butene-1,4-diamine) and PG-11048 ((2E)-N1,N4-bis[3-(ethylamino)propyl]-2-butene-1,4-diamine) in combination with DNA methyltransferase (DNMT) inhibitors, histone deacetylase (HDAC) inhibitors, or both DNA methyltransferase inhibitors and histone deacetylase inhibitors, are disclosed. Hematopoietic cancers, lung cancers, mesothelioma, cutaneous T-cell lymphoma (CTCL), multiple myeloma, solid tumors, and blood disorders such as myelodysplastic syndromes can be treated using the methods of the invention.

### CROSS-REFERENCE TO RELATED APPLICATIONS

This patent application claims priority benefit of U.S. Provisional Patent Application No. 61/300,698, filed Feb. 2, 2010. The entire contents of that application are hereby incorporated by reference herein.

### STATEMENT REGARDING FEDERALLY SPONSORED RESEARCH OR DEVELOPMENT

This invention was made with U.S. Government support under grant NIH NCI CA51085 from the National Cancer Institute of the National Institutes of Health. The Government has certain rights in this invention.

### TECHNICAL FIELD OF THE INVENTION

The present invention relates to formulations of and use of (2Z)-N1,N4-bis[3-(ethylamino) propyl]-2-butene-1,4-diamine (PG-11047) and (2E)-N1,N4-bis[3-(ethylamino) propyl]-2-butene-1,4-diamine (PG-11048) in combination with specific epigenetically-acting pharmaceuticals, such as DNA methyltransferase inhibitors and histone deacetylase inhibitors, for the treatment of cancer and blood disorders.

### BACKGROUND OF THE INVENTION

Cancer continues to be a significant health problem in both developed and developing nations. According to the World Health Organization, 7.4 million deaths (13% of all deaths) worldwide were due to cancer in 2004, and WHO projects that this number will rise to 12 million deaths by 2030.

Dramatic strides have been made in understanding the genetic basis of cancer. Much research has been focused on specific genes that play a role in development of cancer. Oncogenes are genes that, when overexpressed, result in cancer, while tumor suppressor genes are genes that, when underexpressed, result in cancer.

Recently, attention has been directed to epigenetic factors in cancer. Epigenetics deals with phenotypes that arise due to factors other than the DNA sequence of a cell or organism. For example, acetylation of lysine residues on histone proteins (by histone acetyltransferases, or HATs) neutralizes the positive charge on the lysine side chain, reducing the charge-based interaction of the histone protein with the DNA it supports. This loosens the DNA-histone complex, resulting in increased expression of the gene at that location. De-acetylation of the lysine by histone deacetylases (HDACs) restores the strong interaction between histone and DNA, downregulating the gene at that location. If the HDAC enzymes deacetylate histones interacting with a tumor suppressor gene, silencing of that gene can lead to uncontrolled cell proliferation and cancer. DNA methyltransferases (DNA methylases) catalyze transfer of methyl groups onto DNA strands, silencing the gene where the methylation occurs, which is an undesirable occurrence for a tumor suppressor gene.

Previously developed epigenetic-targeting drugs, such as DNA methyltransferase (DNMT) inhibitors and histone deacetylase (HDAC) inhibitors, have had limited success in the clinical setting partially due to dose-limiting toxicities. Some studies have shown, however, that low doses of the two classes of inhibitors (HDAC and DNMT) can be combined to produce a synergistic anti-tumor effect, and various combinations of these inhibitors are now in clinical trials.

The current invention describes specific compounds in combination with pharmaceuticals having an epigenetic effect, such as DNA methyltransferase inhibitors, histone deacetylase inhibitors, or both DNA methyltransferase inhibitors and histone deacetylase inhibitors. One such compound is (2Z)-N1,N4-bis[3-(ethylamino)propyl]-2-butene-1,4-diamine, which is named PG-11047, and also known as SL-11047 and CGC-11047. PG-11047 is a conformationally restricted polyamine analog and is described in U.S. Pat. No. 5,889,061. Another such compound is (2E)-N1,N4-bis[3-(ethylamino)propyl]-2-butene-1,4-diamine, which is referred to herein as PG-11048. PG-11048 is described in WO 95/18091 and US 5,627,215. Using PG-11047 or PG-11048 in combination with DNMT, HDAC, or DNMT and HDAC inhibitors potentiates the effect of the DNMT, HDAC, or DNMT and HDAC inhibitors on cancer cell lines.

### BRIEF SUMMARY OF THE INVENTION

The present invention provides methods for the treatment of proliferative diseases, such as cancer, and methods for the treatment of blood disorders, such as myelodysplastic syndromes (MDS). The invention provides combination therapy methods of treating proliferative diseases (such as cancer) and combination therapy methods of treating blood disorders (such as myelodysplastic syndromes), comprising a) using a first therapy comprising administering to an individual an effective amount of a composition comprising an epigenetically-acting agent and b) using a second therapy comprising administering a chemotherapeutic agent. In some variations, the invention provides combination therapy methods of treating proliferative diseases (such as cancer) and combination therapy methods of treating blood disorders (such as myelodysplastic syndromes), comprising a) using a first therapy comprising administering to an individual an effective amount of a composition comprising an epigenetically-acting agent and b) using a second therapy comprising administering an agent that potentiates or acts synergistically with the epigenetically-acting agent.

In some variations, the epigenetic agent is any of (and in some variations selected from the group consisting of) a DNA methyltransferase inhibitor, a histone deacetylase (HDAC) inhibitor, or a combination of a DNA methyltransferase inhibitor and a histone deacetylase (HDAC) inhibitor.

In some variations, the DNA methyltransferase inhibitor is any of (and in some variations selected from the group consisting of) 5-azacytidine (azacytidine, Azacitidine, 4-amino-1-(3-D-ribofuranosyl-s-triazin-2(1H)-one, Vidaza), decitabine (Dacogen), Zebularine, SGI-110, RG108, or DZNep (SGI-1036, 3-deazaneplanocin A). In some variations, the histone deacetylase inhibitor is any of (and in some variations selected from the group consisting of) sodium phenylbutyrate (Ammonaps, Buphenyl), valproic acid (Depakote), vorinostat (N-hydroxy-N′ -phenyloctanediamide, Zolinza, SAHA), panobinostat (LBH589), belinostat (PXD101), JNJ-26481585, romidepsin (FK228), entinostat (MS-275, SNDX-275), or MGCD-0103.

In some variations, the chemotherapeutic agent is any of (and in some variations selected from the group consisting of) PG-11047 and PG-11048.

In some variations, the agent that potentiates or acts synergistically with the epigenetically-acting agent is any of (and in some variations selected from the group consisting of) PG-11047 and PG-11048.

In some variations, the invention provides a method of treating a proliferative disease (such as cancer) or blood disorders (such as myelodysplastic syndromes) in an individual comprising administering to the individual a) an effective amount of a composition comprising an epigenetically-acting agent and b) an effective amount of an agent selected from PG-11047 and PG-11048. In some variations, the agent selected from PG-11047 and PG-11048 potentiates or acts synergistically with the epigenetically-acting agent.

In some variations, the cancer can be a hematopoietic cancer. In some variations, the hematopoietic cancer is acute myeloid leukemia. In some variations, the cancer is lung cancer. In some variations, the cancer is mesothelioma. In some variations, the cancer is a solid tumor. In some variations, the cancer is cutaneous T-cell lymphoma (CTCL). In some variations, the cancer is multiple myeloma.

In some variations, the blood disorder is a myelodysplastic syndrome. In some variations, the myelodysplastic syndrome (MDS) is refractory anemia. In some variations, the myelodysplastic syndrome (MDS) is refractory anemia with ringed sideroblasts. In some variations, the myelodysplastic syndrome (MDS) is refractory anemia with excess blasts. In some variations, the myelodysplastic syndrome (MDS) is refractory anemia with excess blasts in transformation. In some variations, the myelodysplastic syndrome (MDS) is refractory cytopenia with multilineage dysplasia. In some variations, the myelodysplastic syndrome (MDS) is myelodysplastic syndrome associated with an isolated del(5q) chromosome abnormality. In some variations, the myelodysplastic syndrome (MDS) is unclassifiable myelodysplastic syndrome.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising an epigenetically acting agent, and b) an effective amount of PG-11047. In some variations, PG-11047 potentiates or acts synergistically with the epigenetically-acting agent.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising an epigenetically acting agent, and b) an effective amount of PG-11047. In some variations, PG-11047 potentiates or acts synergistically with the epigenetically-acting agent.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising an epigenetically acting agent, and b) an effective amount of PG-11047. In some variations, PG-11047 potentiates or acts synergistically with the epigenetically-acting agent.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising an epigenetically acting agent, and b) an effective amount of PG-11047. In some variations, PG-11047 potentiates or acts synergistically with the epigenetically-acting agent.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising an epigenetically acting agent, and b) an effective amount of PG-11048. In some variations, PG-11048 potentiates or acts synergistically with the epigenetically-acting agent.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising an epigenetically acting agent, and b) an effective amount of PG-11048. In some variations, PG-11048 potentiates or acts synergistically with the epigenetically-acting agent.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising an epigenetically acting agent, and b) an effective amount of PG-11048. In some variations, PG-11048 potentiates or acts synergistically with the epigenetically-acting agent.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising an epigenetically acting agent, and b) an effective amount of PG-11048. In some variations, PG-11048 potentiates or acts synergistically with the epigenetically-acting agent.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising 5-azacytidine, and b) an effective amount of PG-11047. In some variations, PG-11047 potentiates or acts synergistically with the 5-azacytidine.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising 5-azacytidine, and b) an effective amount of PG-11047. In some variations, PG-11047 potentiates or acts synergistically with the 5-azacytidine.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising 5-azacytidine, and b) an effective amount of PG-11047. In some variations, PG-11047 potentiates or acts synergistically with the 5-azacytidine.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising 5-azacytidine, and b) an effective amount of PG-11047. In some variations, PG-11047 potentiates or acts synergistically with the 5-azacytidine.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising 5-azacytidine, and b) an effective amount of PG-11048. In some variations, PG-11048 potentiates or acts synergistically with the 5-azacytidine.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising 5-azacytidine, and b) an effective amount of PG-11048. In some variations, PG-11048 potentiates or acts synergistically with the 5-azacytidine.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising 5-azacytidine, and b) an effective amount of PG-11048. In some variations, PG-11048 potentiates or acts synergistically with the 5-azacytidine.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising 5-azacytidine, and b) an effective amount of PG-11048. In some variations, PG-11048 potentiates or acts synergistically with the 5-azacytidine.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising decitabine, and b) an effective amount of PG-11047. In some variations, PG-11047 potentiates or acts synergistically with the decitabine.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising decitabine, and b) an effective amount of PG-11047. In some variations, PG-11047 potentiates or acts synergistically with the decitabine.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising decitabine, and b) an effective amount of PG-11047. In some variations, PG-11047 potentiates or acts synergistically with the decitabine.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising decitabine, and b) an effective amount of PG-11047. In some variations, PG-11047 potentiates or acts synergistically with the decitabine.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising decitabine, and b) an effective amount of PG-11048. In some variations, PG-11048 potentiates or acts synergistically with the decitabine.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising decitabine, and b) an effective amount of PG-11048. In some variations, PG-11048 potentiates or acts synergistically with the decitabine.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising decitabine, and b) an effective amount of PG-11048. In some variations, PG-11048 potentiates or acts synergistically with the decitabine.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising decitabine, and b) an effective amount of PG-11048. In some variations, PG-11048 potentiates or acts synergistically with the decitabine.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising Zebularine, and b) an effective amount of PG-11047. In some variations, PG-11047 potentiates or acts synergistically with the Zebularine.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising Zebularine, and b) an effective amount of PG-11047. In some variations, PG-11047 potentiates or acts synergistically with the Zebularine.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising Zebularine, and b) an effective amount of PG-11047. In some variations, PG-11047 potentiates or acts synergistically with the Zebularine.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising Zebularine, and b) an effective amount of PG-11047. In some variations, PG-11047 potentiates or acts synergistically with the Zebularine.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising Zebularine, and b) an effective amount of PG-11048. In some variations, PG-11048 potentiates or acts synergistically with the Zebularine.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising Zebularine, and b) an effective amount of PG-11048. In some variations, PG-11048 potentiates or acts synergistically with the Zebularine.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising Zebularine, and b) an effective amount of PG-11048. In some variations, PG-11048 potentiates or acts synergistically with the Zebularine.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising Zebularine, and b) an effective amount of PG-11048. In some variations, PG-11048 potentiates or acts synergistically with the Zebularine.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising SGI-110, and b) an effective amount of PG-11047. In some variations, PG-11047 potentiates or acts synergistically with the SGI-110.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising SGI-110, and b) an effective amount of PG-11047. In some variations, PG-11047 potentiates or acts synergistically with the SGI-110.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising SGI-110, and b) an effective amount of PG-11047. In some variations, PG-11047 potentiates or acts synergistically with the SGI-110.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising SGI-110, and b) an effective amount of PG-11047. In some variations, PG-11047 potentiates or acts synergistically with the SGI-110.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising SGI-110, and b) an effective amount of PG-11048. In some variations, PG-11048 potentiates or acts synergistically with the SGI-110.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising SGI-110, and b) an effective amount of PG-11048. In some variations, PG-11048 potentiates or acts synergistically with the SGI-110.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising SGI-110, and b) an effective amount of PG-11048. In some variations, PG-11048 potentiates or acts synergistically with the SGI-110.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising SGI-110, and b) an effective amount of PG-11048. In some variations, PG-11048 potentiates or acts synergistically with the SGI-110.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising RG108, and b) an effective amount of PG-11047. In some variations, PG-11047 potentiates or acts synergistically with the RG108.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising RG108, and b) an effective amount of PG-11047. In some variations, PG-11047 potentiates or acts synergistically with the RG108.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising RG108, and b) an effective amount of PG-11047. In some variations, PG-11047 potentiates or acts synergistically with the RG108.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising RG108, and b) an effective amount of PG-11047. In some variations, PG-11047 potentiates or acts synergistically with the RG108.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising RG108, and b) an effective amount of PG-11048. In some variations, PG-11048 potentiates or acts synergistically with the RG108.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising RG108, and b) an effective amount of PG-11048. In some variations, PG-11048 potentiates or acts synergistically with the RG108.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising RG108, and b) an effective amount of PG-11048. In some variations, PG-11048 potentiates or acts synergistically with the RG108.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising RG108, and b) an effective amount of PG-11048. In some variations, PG-11048 potentiates or acts synergistically with the RG108.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising DZNep, and b) an effective amount of PG-11047. In some variations, PG-11047 potentiates or acts synergistically with the DZNep.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising DZNep, and b) an effective amount of PG-11047. In some variations, PG-11047 potentiates or acts synergistically with the DZNep.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising DZNep, and b) an effective amount of PG-11047. In some variations, PG-11047 potentiates or acts synergistically with the DZNep.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising DZNep, and b) an effective amount of PG-11047. In some variations, PG-11047 potentiates or acts synergistically with the DZNep.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising DZNep, and b) an effective amount of PG-11048. In some variations, PG-11048 potentiates or acts synergistically with the DZNep.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising DZNep, and b) an effective amount of PG-11048. In some variations, PG-11048 potentiates or acts synergistically with the DZNep.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising DZNep, and b) an effective amount of PG-11048. In some variations, PG-11048 potentiates or acts synergistically with the DZNep.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising DZNep, and b) an effective amount of PG-11048. In some variations, PG-11048 potentiates or acts synergistically with the DZNep.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising sodium phenylbutyrate, and b) an effective amount of PG-11047. In some variations, PG-11047 potentiates or acts synergistically with the sodium phenylbutyrate.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising sodium phenylbutyrate, and b) an effective amount of PG-11047. In some variations, PG-11047 potentiates or acts synergistically with the sodium phenylbutyrate.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising sodium phenylbutyrate, and b) an effective amount of PG-11047. In some variations, PG-11047 potentiates or acts synergistically with the sodium phenylbutyrate.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising sodium phenylbutyrate, and b) an effective amount of PG-11047. In some variations, PG-11047 potentiates or acts synergistically with the sodium phenylbutyrate.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising sodium phenylbutyrate, and b) an effective amount of PG-11048. In some variations, PG-11048 potentiates or acts synergistically with the sodium phenylbutyrate.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising sodium phenylbutyrate, and b) an effective amount of PG-11048. In some variations, PG-11048 potentiates or acts synergistically with the sodium phenylbutyrate.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising sodium phenylbutyrate, and b) an effective amount of PG-11048. In some variations, PG-11048 potentiates or acts synergistically with the sodium phenylbutyrate.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising sodium phenylbutyrate, and b) an effective amount of PG-11048. In some variations, PG-11048 potentiates or acts synergistically with the sodium phenylbutyrate.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising valproic acid, and b) an effective amount of PG-11047. In some variations, PG-11047 potentiates or acts synergistically with the valproic acid.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising valproic acid, and b) an effective amount of PG-11047. In some variations, PG-11047 potentiates or acts synergistically with the valproic acid.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising valproic acid, and b) an effective amount of PG-11047. In some variations, PG-11047 potentiates or acts synergistically with the valproic acid.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising valproic acid, and b) an effective amount of PG-11047. In some variations, PG-11047 potentiates or acts synergistically with the valproic acid.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising valproic acid, and b) an effective amount of PG-11048. In some variations, PG-11048 potentiates or acts synergistically with the valproic acid.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising valproic acid, and b) an effective amount of PG-11048. In some variations, PG-11048 potentiates or acts synergistically with the valproic acid.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising valproic acid, and b) an effective amount of PG-11048. In some variations, PG-11048 potentiates or acts synergistically with the valproic acid.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising valproic acid, and b) an effective amount of PG-11048. In some variations, PG-11048 potentiates or acts synergistically with the valproic acid.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising vorinostat, and b) an effective amount of PG-11047. In some variations, PG-11047 potentiates or acts synergistically with the vorinostat.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising vorinostat, and b) an effective amount of PG-11047. In some variations, PG-11047 potentiates or acts synergistically with the vorinostat.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising vorinostat, and b) an effective amount of PG-11047. In some variations, PG-11047 potentiates or acts synergistically with the vorinostat.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising vorinostat, and b) an effective amount of PG-11047. In some variations, PG-11047 potentiates or acts synergistically with the vorinostat.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising vorinostat, and b) an effective amount of PG-11048. In some variations, PG-11048 potentiates or acts synergistically with the vorinostat.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising vorinostat, and b) an effective amount of PG-11048. In some variations, PG-11048 potentiates or acts synergistically with the vorinostat.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising vorinostat, and b) an effective amount of PG-11048. In some variations, PG-11048 potentiates or acts synergistically with the vorinostat.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising vorinostat, and b) an effective amount of PG-11048. In some variations, PG-11048 potentiates or acts synergistically with the vorinostat.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising panobinostat, and b) an effective amount of PG-11047. In some variations, PG-11047 potentiates or acts synergistically with the panobinostat.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising panobinostat, and b) an effective amount of PG-11047. In some variations, PG-11047 potentiates or acts synergistically with the panobinostat.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising panobinostat, and b) an effective amount of PG-11047. In some variations, PG-11047 potentiates or acts synergistically with the panobinostat.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising panobinostat, and b) an effective amount of PG-11047. In some variations, PG-11047 potentiates or acts synergistically with the panobinostat.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising panobinostat, and b) an effective amount of PG-11048. In some variations, PG-11048 potentiates or acts synergistically with the panobinostat.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising panobinostat, and b) an effective amount of PG-11048. In some variations, PG-11048 potentiates or acts synergistically with the panobinostat.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising panobinostat, and b) an effective amount of PG-11048. In some variations, PG-11048 potentiates or acts synergistically with the panobinostat.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising panobinostat, and b) an effective amount of PG-11048. In some variations, PG-11048 potentiates or acts synergistically with the panobinostat.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising belinostat, and b) an effective amount of PG-11047. In some variations, PG-11047 potentiates or acts synergistically with the belinostat.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising belinostat, and b) an effective amount of PG-11047. In some variations, PG-11047 potentiates or acts synergistically with the belinostat.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising belinostat, and b) an effective amount of PG-11047. In some variations, PG-11047 potentiates or acts synergistically with the belinostat.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising belinostat, and b) an effective amount of PG-11047. In some variations, PG-11047 potentiates or acts synergistically with the belinostat.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising belinostat, and b) an effective amount of PG-11048. In some variations, PG-11048 potentiates or acts synergistically with the belinostat.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising belinostat, and b) an effective amount of PG-11048. In some variations, PG-11048 potentiates or acts synergistically with the belinostat.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising belinostat, and b) an effective amount of PG-11048. In some variations, PG-11048 potentiates or acts synergistically with the belinostat.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising belinostat, and b) an effective amount of PG-11048. In some variations, PG-11048 potentiates or acts synergistically with the belinostat.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising JNJ-26481585, and b) an effective amount of PG-11047. In some variations, PG-11047 potentiates or acts synergistically with the JNJ-26481585.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising JNJ-26481585, and b) an effective amount of PG-11047. In some variations, PG-11047 potentiates or acts synergistically with the JNJ-26481585.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising JNJ-26481585, and b) an effective amount of PG-11047. In some variations, PG-11047 potentiates or acts synergistically with the JNJ-26481585.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising JNJ-26481585, and b) an effective amount of PG-11047. In some variations, PG-11047 potentiates or acts synergistically with the JNJ-26481585.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising JNJ-26481585, and b) an effective amount of PG-11048. In some variations, PG-11048 potentiates or acts synergistically with the JNJ-26481585.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising JNJ-26481585, and b) an effective amount of PG-11048. In some variations, PG-11048 potentiates or acts synergistically with the JNJ-26481585.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising JNJ-26481585, and b) an effective amount of PG-11048. In some variations, PG-11048 potentiates or acts synergistically with the JNJ-26481585.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising JNJ-26481585, and b) an effective amount of PG-11048. In some variations, PG-11048 potentiates or acts synergistically with the JNJ-26481585.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising romidepsin, and b) an effective amount of PG-11047. In some variations, PG-11047 potentiates or acts synergistically with the romidepsin.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising romidepsin, and b) an effective amount of PG-11047. In some variations, PG-11047 potentiates or acts synergistically with the romidepsin.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising romidepsin, and b) an effective amount of PG-11047. In some variations, PG-11047 potentiates or acts synergistically with the romidepsin.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising romidepsin, and b) an effective amount of PG-11047. In some variations, PG-11047 potentiates or acts synergistically with the romidepsin.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising romidepsin, and b) an effective amount of PG-11048. In some variations, PG-11048 potentiates or acts synergistically with the romidepsin.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising romidepsin, and b) an effective amount of PG-11048. In some variations, PG-11048 potentiates or acts synergistically with the romidepsin.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising romidepsin, and b) an effective amount of PG-11048. In some variations, PG-11048 potentiates or acts synergistically with the romidepsin.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising romidepsin, and b) an effective amount of PG-11048. In some variations, PG-11048 potentiates or acts synergistically with the romidepsin.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising entinostat, and b) an effective amount of PG-11047. In some variations, PG-11047 potentiates or acts synergistically with the entinostat.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising entinostat, and b) an effective amount of PG-11047. In some variations, PG-11047 potentiates or acts synergistically with the entinostat.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising entinostat, and b) an effective amount of PG-11047. In some variations, PG-11047 potentiates or acts synergistically with the entinostat.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising entinostat, and b) an effective amount of PG-11047. In some variations, PG-11047 potentiates or acts synergistically with the entinostat.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising entinostat, and b) an effective amount of PG-11048. In some variations, PG-11048 potentiates or acts synergistically with the entinostat.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising entinostat, and b) an effective amount of PG-11048. In some variations, PG-11048 potentiates or acts synergistically with the entinostat.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising entinostat, and b) an effective amount of PG-11048. In some variations, PG-11048 potentiates or acts synergistically with the entinostat.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising entinostat, and b) an effective amount of PG-11048. In some variations, PG-11048 potentiates or acts synergistically with the entinostat.

In some variations, there is provided a method of treating a disease such as cancer (for example hematopoietic cancers, lung cancer, mesothelioma, cutaneous T-cell lymphoma (CTCL), multiple myeloma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising MGCD-0103, and b) an effective amount of PG-11047. In some variations, PG-11047 potentiates or acts synergistically with the MGCD-0103.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising MGCD-0103, and b) an effective amount of PG-11047. In some variations, PG-11047 potentiates or acts synergistically with the MGCD-0103.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising MGCD-0103, and b) an effective amount of PG-11047. In some variations, PG-11047 potentiates or acts synergistically with the MGCD-0103.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising MGCD-0103, and b) an effective amount of PG-11047. In some variations, PG-11047 potentiates or acts synergistically with the MGCD-0103.

In some variations, there is provided a method of treating a disease such as cancer (for example hematopoietic cancers, lung cancer, cutaneous T-cell lymphoma (CTCL), multiple myeloma, mesothelioma, or solid tumors), or a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising MGCD-0103, and b) an effective amount of PG-11048. In some variations, PG-11048 potentiates or acts synergistically with the MGCD-0103.

In some variations, there is provided a method of treating cancer (for example hematopoietic cancers, lung cancer, mesothelioma, cutaneous T-cell lymphoma (CTCL), multiple myeloma, or solid tumors) in an individual, comprising administering to the individual: a) an effective amount of a composition comprising MGCD-0103, and b) an effective amount of PG-11048. In some variations, PG-11048 potentiates or acts synergistically with the MGCD-0103.

In some variations, there is provided a method of treating a blood disorder (such as myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome) in an individual, comprising administering to the individual: a) an effective amount of a composition comprising MGCD-0103, and b) an effective amount of PG-11048. In some variations, PG-11048 potentiates or acts synergistically with the MGCD-0103.

In some variations, there is provided a method of treating myelodysplastic syndrome (MDS), for example refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome, in an individual, comprising administering to the individual: a) an effective amount of a composition comprising MGCD-0103, and b) an effective amount of PG-11048. In some variations, PG-11048 potentiates or acts synergistically with the MGCD-0103.

In some variations, the composition comprising the epigenetically active agent and the PG-11047 or PG-11048 can be administered simultaneously, either in the same composition or in separate compositions. In some variations, the epigenetically active agent composition and the PG-11047 or PG-11048 are administered sequentially, i.e., the epigenetically active agent composition is administered either prior to or after the administration of PG-11047 or PG-11048. In some variations, the administration of the epigenetically active agent composition and the PG-11047 or PG-11048 are concurrent, i.e., the administration period of the epigenetically active agent composition and the PG-11047 or PG-11048 overlap with each other. In some variations, the administration of the epigenetically active agent composition and the PG-11047 or PG-11048 are non-concurrent. For example, in some variations, the administration of the epigenetically active agent composition is terminated before the PG-11047 or PG-11048 is administered. In some variations, the administration of PG-11047 or PG-11048 is terminated before the epigenetically active agent composition is administered.

The invention provides combinations of PG-11047 with epigenetically-acting drugs, such as epigenetically-acting chemotherapeutic drugs, in synergistic amounts. In one embodiment, the synergistic combinations of PG-11047 with one or more epigenetically-acting drugs, such as one or more epigenetically-acting chemotherapeutic drugs, comprises a pharmaceutically acceptable composition comprising PG-11047 and an epigenetically-acting drug, such as an epigenetically-acting chemotherapeutic drug, in synergistic amounts. In one embodiment, the synergistic combination comprises a pharmaceutically acceptable composition comprising PG-11047 and a DNA methyltransferase inhibitor in synergistic amounts. In another embodiment, the synergistic combination comprises a pharmaceutically acceptable composition comprising PG-11047, a DNA methyltransferase inhibitor, and one or more additional drugs in synergistic amounts. In another embodiment, the synergistic combination comprises a pharmaceutically acceptable composition comprising PG-11047 and a histone deacetylase (HDAC) inhibitor in synergistic amounts. In another embodiment, the synergistic combination comprises a pharmaceutically acceptable composition comprising PG-11047, a histone deacetylase (HDAC) inhibitor, and one or more additional drugs in synergistic amounts. In another embodiment, the synergistic combination comprises a pharmaceutically acceptable composition comprising PG-11047, a DNA methyltransferase inhibitor and a histone deacetylase (HDAC) inhibitor in synergistic amounts. In another embodiment, the synergistic combination comprises a pharmaceutically acceptable composition comprising PG-11047, a DNA methyltransferase inhibitor, a histone deacetylase (HDAC) inhibitor, and one or more additional drugs in synergistic amounts.

In one embodiment, the synergistic combination of PG-11047 with a DNA methyltransferase inhibitor comprises a pharmaceutically acceptable composition comprising PG-11047 and 5-azacytidine in synergistic amounts. In another embodiment, the synergistic combination of PG-11047 with a DNA methyltransferase inhibitor comprises a pharmaceutically acceptable composition comprising PG-11047 and decitabine in synergistic amounts. In another embodiment, the synergistic combination of PG-11047 with a DNA methyltransferase inhibitor comprises a pharmaceutically acceptable composition comprising PG-11047 and Zebularine in synergistic amounts. In another embodiment, the synergistic combination of PG-11047 with a DNA methyltransferase inhibitor comprises a pharmaceutically acceptable composition comprising PG-11047 and SGI-110 in synergistic amounts. In another embodiment, the synergistic combination of PG-11047 with a DNA methyltransferase inhibitor comprises a pharmaceutically acceptable composition comprising PG-11047 and RG108 in synergistic amounts. In another embodiment, the synergistic combination of PG-11047 with a DNA methyltransferase inhibitor comprises a pharmaceutically acceptable composition comprising PG-11047 and DZNep in synergistic amounts.

In one embodiment, the synergistic combination of PG-11047 with a histone deacetylase (HDAC) inhibitor comprises a pharmaceutically acceptable composition comprising PG-11047 and sodium phenylbutyrate in synergistic amounts. In another embodiment, the synergistic combination of PG-11047 with a histone deacetylase (HDAC) inhibitor comprises a pharmaceutically acceptable composition comprising PG-11047 and valproic acid in synergistic amounts. In another embodiment, the synergistic combination of PG-11047 with a histone deacetylase (HDAC) inhibitor comprises a pharmaceutically acceptable composition comprising PG-11047 and vorinostat in synergistic amounts. In another embodiment, the synergistic combination of PG-11047 with a histone deacetylase (HDAC) inhibitor comprises a pharmaceutically acceptable composition comprising PG-11047 and panobinostat in synergistic amounts. In another embodiment, the synergistic combination of PG-11047 with a histone deacetylase (HDAC) inhibitor comprises PG-11047 and a pharmaceutically acceptable composition comprising belinostat in synergistic amounts. In another embodiment, the synergistic combination of PG-11047 with a histone deacetylase (HDAC) inhibitor comprises a pharmaceutically acceptable composition comprising PG-11047 and JNJ-26481585 in synergistic amounts. In another embodiment, the synergistic combination of PG-11047 with a histone deacetylase (HDAC) inhibitor comprises a pharmaceutically acceptable composition comprising PG-11047 and romidepsin in synergistic amounts. In another embodiment, the synergistic combination of PG-11047 with a histone deacetylase (HDAC) inhibitor comprises a pharmaceutically acceptable composition comprising PG-11047 and entinostat in synergistic amounts. In another embodiment, the synergistic combination of PG-11047 with a histone deacetylase (HDAC) inhibitor comprises a pharmaceutically acceptable composition comprising PG-11047 and MGCD-0103 in synergistic amounts.

In another embodiment, the synergistic combination comprises a pharmaceutically acceptable composition comprising PG-11047, vorinostat (SAHA) and 5-azacytidine in synergistic amounts. In another embodiment, the synergistic combination comprises a pharmaceutically acceptable composition comprising PG-11047, vorinostat (SAHA) and decitabine in synergistic amounts. In another embodiment, the synergistic combination comprises a pharmaceutically acceptable composition comprising PG-11047, vorinostat (SAHA) and NPI-0052 (Salinosporamide A) in synergistic amounts. In another embodiment, the synergistic combination comprises a pharmaceutically acceptable composition comprising PG-11047, entinostat (MS-275, SNDX-275) and 5-azacytidine in synergistic amounts. In another embodiment, the synergistic combination comprises a pharmaceutically acceptable composition comprising PG-11047, panobinostat (LBH589) and decitabine in synergistic amounts. In another embodiment, the synergistic combination comprises a pharmaceutically acceptable composition comprising PG-11047, valproic acid and 5-azacytidine in synergistic amounts. In another embodiment, the synergistic combination comprises a pharmaceutically acceptable composition comprising PG-11047, valproic acid and decitabine in synergistic amounts. In another embodiment, the synergistic combination comprises a pharmaceutically acceptable composition comprising PG-11047, valproic acid, 5-azacytidine, and ATRA (all-trans retinoic acid) in synergistic amounts. In another embodiment, the synergistic combination comprises a pharmaceutically acceptable composition comprising PG-11047, belinostat and 5-azacytidine in synergistic amounts. In another embodiment, the synergistic combination comprises a pharmaceutically acceptable composition comprising PG-11047, MGCD-0103 and 5-azacytidine in synergistic amounts.

In additional embodiments, the invention embraces a method of treating cancer, comprising administering one or more of the any of the compositions described above, to a patient having cancer. The cancer can be selected from the group comprising hematopoietic cancers, myelodysplastic syndrome, acute myeloid leukemia, lung cancers, mesothelioma, cutaneous T-cell lymphoma (CTCL), multiple myeloma, and solid tumors. In another embodiment, the cancer is selected from hematopoietic cancers. In another embodiment, the cancer is myelodysplastic syndrome. In another embodiment, the cancer is acute myeloid leukemia. In another embodiment, the cancer is lung cancer. In another embodiment, the cancer is mesothelioma. In another embodiment, the cancer is cutaneous T-cell lymphoma (CTCL). In another embodiment, the cancer is multiple myeloma. In another embodiment, the cancer is a solid tumor.

### BRIEF DESCRIPTION OF THE DRAWINGS

FIG. 1 shows global changes in H3K4me2 and AcH3K9 proteins (i.e., global changes in activating histone marks) in Calu-6 following 24 hr or 48 hr treatments with PG-11047. 30 μg of nuclear protein are loaded per lane.

FIG. 2 shows gene expression (measured by qPCR) in Calu-6 cells in the absence of, or in the presence of varying concentrations of, PG-11047, of CDH-13, p16, sFRP2, and GATA-4.

FIG. 3A shows quantitative PCR of CDH-13 gene expression after 24 h combination treatments of Calu-6 cells at various concentrations of PG-11047 with varying concentrations of 5-AC and MS-275. Values represent fold increase in expression of treated cells as compared to untreated Calu-6. Asterisks indicate synergistic or additive effect.

FIG. 3B shows quantitative PCR of sFRP2 gene expression after 24 h combination treatments of Calu-6 cells at various concentrations of PG-11047 with varying concentrations of 5-AC and MS-275. Values represent percent expression of Calu-6 cells treated with 1 μM 5-azacytidine alone (i.e., expression of Calu-6 cells treated with 1 μM 5-azacytidine alone is set to 100); an accurate quantification of untreated Calu-6 was not possible due to extremely low endogenous sFRP2 message. Asterisks indicate synergistic or additive effect.

FIG. 3C shows quantitative PCR of p16 gene expression after 24 h combination treatments of Calu-6 cells at various concentrations of PG-11047 with varying concentrations of 5-AC and MS-275. Values represent fold increase in expression of treated cells as compared to untreated Calu-6. Asterisks indicate synergistic or additive effect.

FIG. 3D shows quantitative PCR of GATA-4 gene expression after 24 h combination treatments of Calu-6 cells at various concentrations of PG-11047 with varying concentrations of 5-AC and MS-275. Values represent fold increase in expression of treated cells as compared to untreated Calu-6. Asterisks indicate synergistic or additive effect.

FIG. 4A shows quantitative PCR of CDH-13 gene expression after 24 h combination treatments of Calu-6 cells at various concentrations of PG-11048 with varying concentrations of 5-AC and MS-275. Values represent fold increase in expression of treated cells as compared to untreated Calu-6. Asterisks indicate synergistic or additive effect.

FIG. 4B shows quantitative PCR of sFRP2 gene expression after 24 h combination treatments of Calu-6 cells at various concentrations of PG-11048 with varying concentrations of 5-AC and MS-275. Values represent percent expression of Calu-6 cells treated with 1 μM 5-azacytidine alone (i.e., expression of Calu-6 cells treated with 1 μM 5-azacytidine alone is set to 100). Asterisks indicate synergistic or additive effect.

FIG. 4C shows quantitative PCR of GATA-4 gene expression after 24 h combination treatments of Calu-6 cells at various concentrations of PG-11048 with varying concentrations of 5-AC and MS-275. Values represent fold increase in expression of treated cells as compared to untreated Calu-6. Asterisks indicate synergistic or additive effect.

FIG. 4D shows quantitative PCR of p16 gene expression after 24 h combination treatments of Calu-6 cells at various concentrations of PG-11048 with varying concentrations of 5-AC and MS-275. Values represent fold increase in expression of treated cells as compared to untreated Calu-6. Asterisks indicate synergistic or additive effect.

FIG. 5 shows the results of treatment with control, 5-azacytidine (0.5 mg/kg/day) alone, PG-11047 (100 mg/kg/week) alone, and with the combination of 5-azacytidine and PG-11047 on tumor volume in mice.

FIG. 6 shows the results of treatment with control, 5-azacytidine (0.5 mg/kg/day) alone, PG-11047 (100 mg/kg/week) alone, and with the combination of 5-azacytidine and PG-11047 on body mass in mice

FIG. 7 shows the results of ex vivo treatment of tumor cells with control, 5-azacytidine (500 nM) alone, PG-11047 (5 μM) alone, and with the combination of 5-azacytidine and PG-11047 on subsequent tumor volume after implantation in mice.

### DETAILED DESCRIPTION OF THE INVENTION

The present invention provides methods of combination therapy comprising a first therapy comprising administration of an epigenetically acting chemotherapeutic agent in conjunction with a second chemotherapeutic agent. The second chemotherapeutic agent can be PG-11047 or PG-11048. The second chemotherapeutic agent can potentiate the effect of the epigenetically acting chemotherapeutic agent, or act synergistically with the epigenetically acting chemotherapeutic agent. The present invention involves the discovery that PG-11047 and PG-11048, while having little to no epigenetic activity by themselves, can increase (potentiate) the activity of agents that do have epigenetic activity.

The present invention in one its variations is the use of a first agent comprising an epigenetically-acting agent, such as a DNA methyltransferase inhibitor or a histone deacetylase inhibitor, in combination with a second chemotherapeutic agent or agents. The second chemotherapeutic agent can potentiate or act synergistically with the epigenetically-acting agent. The second chemotherapeutic agent can be PG-11047 or PG-11048. The first agent and second agent can be administered sequentially, or they can be co-administered, and even administered simultaneously in the same pharmaceutical composition or as separate compositions. The reference to “first agent” and “second agent” does not indicate the order of administration of the agents; the first agent can be administered before, during, or after the second agent is administered.

In some variations, the invention provides pharmaceutical compositions comprising an epigenetically-acting agent (such as a DNA methyltransferase inhibitor or a histone deacetylase inhibitor) and a second chemotherapeutic agent that potentiates or acts synergistically with the epigenetically-acting agent for use in the treatment of a cancer, such as a hematopoietic cancer, lung cancer, mesothelioma, cutaneous T-cell lymphoma (CTCL), multiple myeloma, or solid tumor, or for use in the treatment of a blood disorder such as myelodysplastic syndrome, wherein said use comprises simultaneous and/or sequential administration of the epigenetically-acting agent and the second chemotherapeutic agent, where the agents can be administered in any order (that is, the “second” chemotherapeutic agent can be administered before, during, or after administration of the epigenetically-acting agent).

The compound PG-11047 (also known as SL-11047 and CGC-11047) is (2Z)-N1,N4-bis[3-(ethylamino)propyl]-2-butene-1,4-diamine, represented by the following structure:

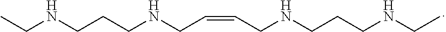 [SMILES: CCNCCCNC/C=C\CNCCCNCC]

The Chemical Abstracts REGISTRY number of PG-11047 is 308145-19-9, while the REGISTRY number of the hydrochloride salt of PG-11047 is 206991-64-2. The synthesis of PG-11047 is described in Reddy VK et al., J. Med. Chem. (1998) 41:4723-4732, and U.S. Pat. No. 5,889,061.

The compound PG-11048 (also known as SL-11048 and CGC-11048) is (2E)-N1,N4-bis[3-(ethylamino)propyl]-2-butene-1,4-diamine, represented by the following structure:

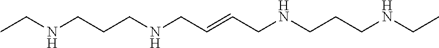 [SMILES: CCNCCCNC/C=C/CNCCCNCC]

The synthesis of PG-11048 is described in WO 95/18091 and U.S. Pat. No. 5,627,215.

The methods described herein are generally useful for treatment of cancers, especially hematopoietic cancers (such as acute myeloid leukemia), lung cancers, mesothelioma, cutaneous T-cell lymphoma (CTCL), multiple myeloma, and solid tumors, and blood disorders such as myelodysplastic syndromes (such as refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome). As used herein, “treatment” is an approach for obtaining beneficial or desired clinical results. For purposes of this invention, beneficial or desired clinical results include, but are not limited to, any one or more of: alleviation of one or more symptoms, diminishment of extent of disease, stabilized (i.e., not worsening) state of disease, preventing or delaying spread (e.g., metastasis) of disease, preventing or delaying occurrence or recurrence of disease, delay or slowing of disease progression, amelioration of the disease state, and remission (whether partial or total). Also encompassed by “treatment” is a reduction of pathological consequence of a disease. The methods of the invention contemplate any one or more of these aspects of treatment.

The term “effective amount” used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some variations, an effective amount is an amount sufficient to delay development. In some variations, an effective amount is an amount sufficient to prevent or delay occurrence and/or recurrence. An effective amount can be administered in one or more administrations. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The term “individual” or “patient” is a mammal, including humans. Individuals or patients include, but are not limited to, humans, bovines, horses, cats, dogs, rodents, or primates.

It is understood that aspects, embodiments, and variations of the invention described herein include “consisting” and/or “consisting essentially of” aspects, embodiments, and variations.

As is understood by one skilled in the art, reference to “about” a value or parameter herein includes (and describes) embodiments that are directed to that value or parameter per se. For example, description referring to “about X” includes description of “X.”

Drug Synergism

A combination of two drugs (or a combination of more than two drugs) acts synergistically when the total effect of the two (or more) drugs combined is greater than the sum of the two (or more) drugs taken independently. That is, two drugs (or a combination of more than two drugs) act synergistically when the total effect of the two (or more) drugs combined is more than a simple additive effect. The synergistically enhanced effect can be any property of the drugs, either alone or in combination. Thus, drugs may act synergistically when lower amounts of one or more drugs in the combination are needed to produce a given effect than would be expected by their additive effects, or when the same amounts of one or more drugs in the combination produce fewer side effects than would be expected by their additive effects, or when the same amounts of one or more drugs in the combination produce a greater effect than would be expected by their additive effects.

Synergistic/Potentiating Effect of PG-11047 and PG-11048 When Administered with DNMT and HDAC Inhibitors

Studies of the effect of PG-11047 and PG-11048 with various chemotherapeutic agents revealed that PG-11047 and PG-11048 are particularly useful in combination with DNA methyltransferase (DNMT) inhibitors and histone deacetylase (HDAC) inhibitors. At certain concentrations, combinations of PG-11047 or PG-11048 with drugs in these classes demonstrated synergistic or potentiating effects on the human Calu-6 lung carcinoma cell line.

Specific DNA methyltransferase (DNMT) inhibitors for combination with PG-11047 or PG-11048 include, but are not limited to: 5-azacytidine (azacytidine, Azacitidine, 4-amino-1-β-D-ribofuranosyl-s-triazin-2(1H)-one, Vidaza), decitabine (5-aza-2′-deoxycytidine, Dacogen), Zebularine (pyrimidin-2-one beta-ribofuranoside), SGI-110 (2′-deoxy-5-azacytidylyl-(3′→5′)-2′-deoxyguanosine), RG108 (N-phthalyl-L-tryptophan), and DZNep (SGI-1036, 3-deazaneplanocin A).

Specific histone deacetylase (HDAC) inhibitors for combination with PG-11047 or PG-11048 include, but are not limited to: sodium phenylbutyrate (Ammonaps, Buphenyl), valproic acid (Depakote), vorinostat (N-hydroxy-N′-phenyloctanediamide, Zolinza, SAHA), panobinostat (LBH589), belinostat (PXD101), JNJ-26481585 (N-hydroxy-2-[4-[[[(1-methyl-1H-indol -3-yl ) methyl]amino]methyl]-1-piperidinyl]-5-pyrimidinecarboxamide), romidepsin (FK228), entinostat (MS-275, SNDX-275), and MGCD-0103 (mocetinostat).

Combinations of drugs which can also be used in combination with PG-11047 or PG-11048 include vorinostat (SAHA) and 5-azacytidine; vorinostat (SAHA) and decitabine; vorinostat (SAHA) and NPI-0052 (Salinosporamide A); entinostat (MS-275, SNDX-275) and 5-azacytidine; panobinostat (LBH589) and decitabine; valproic acid and 5-azacytidine; valproic acid and decitabine; valproic acid, 5-azacytidine, and ATRA (all-trans retinoic acid); belinostat and 5-azacytidine; MGCD-0103 and 5-azacytidine.

Cancers and Diseases Amenable to Treatment With the Combination Therapies of the Invention

The combination therapies are useful in the treatment of cancer, and in the treatment of blood disorders, including diseases that can progress to cancer. Particular cancers amenable to treatment with the combination therapies include hematopoietic cancers, such as acute myeloid leukemia, and blood disorders such as myelodysplastic syndromes. Myelodysplastic syndromes (MDS) include refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, and unclassifiable myelodysplastic syndrome (see World-Wide-Web.cancer.gov/cancertopics/pdq/treatment/myelodysplastic/Patient). MDS is often classified as a pre-cancerous syndrome, as many patients with MDS go on to develop cancer.

The cancer may also be a lung cancer.

The cancer may also be mesothelioma.

The cancer may also be cutaneous T-cell lymphoma (CTCL) (such as mycosis fungoides and/or Sézary syndrome).

The cancer may also be multiple myeloma.

The cancer may also be a solid tumor.

Methods of Administration of Combination Therapies

The combination therapies of the present invention can be administered to a mammalian, preferably human, subject via any route known in the art, including, but not limited to, those disclosed herein. Methods of administration include but are not limited to, oral, intravenous, intraarterial, intratumoral, intramuscular, topical, inhalation, subcutaneous, intraperitoneal, gastrointestinal, and directly to a specific or affected organ. Oral administration in particular is a convenient route for administration, particularly when oral administration provides equivalent therapeutic results as compared with other routes. The combination therapies described herein can be administered in the form of tablets, pills, powder mixtures, capsules, granules, injectables, creams, solutions, suppositories, emulsions, dispersions, food premixes, and in other suitable forms. The combinations can also be administered in liposome formulations. The combinations can also be administered as prodrugs, where the prodrug undergoes transformation in the treated subject to a form which is therapeutically effective. Additional methods of administration are known in the art.

The pharmaceutical dosage form which contains the combinations described herein is conveniently admixed with a pharmaceutically acceptable carrier, such as a non-toxic pharmaceutical organic carrier or a non-toxic pharmaceutical inorganic carrier, in order to provide a pharmaceutically acceptable composition. Typical pharmaceutically-acceptable carriers include, for example, mannitol, urea, dextrans, lactose, potato and maize starches, magnesium stearate, talc, vegetable oils, polyalkylene glycols, ethyl cellulose, poly(vinylpyrrolidone), calcium carbonate, ethyl oleate, isopropyl myristate, benzyl benzoate, sodium carbonate, gelatin, potassium carbonate, silicic acid, and other conventionally employed acceptable carriers. The pharmaceutical dosage form can also contain non-toxic auxiliary substances such as emulsifying, preserving, or wetting agents, and the like. A suitable carrier is one which does not cause an intolerable side effect, but which allows the combination therapies to retain pharmacological activity in the body. Formulations for parenteral and nonparenteral drug delivery are known in the art and are set forth in Remington's Pharmaceutical Sciences, 18th Edition, Mack Publishing (1990). Solid forms, such as tablets, capsules and powders, can be fabricated using conventional tableting and capsule-filling machinery, which is well known in the art. Solid dosage forms, including tablets and capsules for oral administration in unit dose presentation form, can contain any number of additional non-active ingredients known to the art, including such conventional additives as excipients; desiccants; colorants; binding agents, for example syrup, acacia, gelatin, sorbitol, tragacanth, or polyvinylpyrollidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulfate. The tablets can be coated according to methods well known in standard pharmaceutical practice. Liquid forms for ingestion can be formulated using known liquid carriers, including aqueous and non-aqueous carriers, suspensions, oil-in-water and/or water-in-oil emulsions, and the like. Liquid formulations can also contain any number of additional non-active ingredients, including colorants, fragrance, flavorings, viscosity modifiers, preservatives, stabilizers, and the like. For parenteral administration, the combination therapies can be administered as injectable dosages of a solution or suspension of the compound in a physiologically acceptable diluent or sterile liquid carrier such as water or oil, with or without additional surfactants or adjuvants. An illustrative list of carrier oils includes animal and vegetable oils (e.g., peanut oil, soy bean oil), petroleum-derived oils (e.g., mineral oil), and synthetic oils. In general, for injectable unit doses, water, saline, aqueous dextrose and related sugar solutions, and ethanol and glycol solutions such as propylene glycol or polyethylene glycol are preferred liquid carriers. The pharmaceutical unit dosage chosen is preferably fabricated and administered to provide a final concentration of drug at the point of contact with the cancer cell of from, for example, 1 μM to 10 mM or from, for example, 0.1 to 100 μM. The optimal effective concentration of the combination therapy can be determined empirically and will depend on the type and severity of the disease, route of administration, disease progression and health and mass or body area of the patient. Combination therapies can be administered as the sole active agents, or can be administered in combination with one or more active agents, including, but not limited to, cytotoxic agents, antibiotics, antimetabolites, polypeptides, antibodies, cytokines, or one or more additional chemotherapeutic agents.

The chemotherapeutic agents described herein can be the agents themselves, pharmaceutically acceptable salts thereof, and pharmaceutically acceptable esters thereof, as well as stereoisomers, enantiomers, racemic mixtures, and the like. The chemotherapeutic agent or agents as described can be administered as well as a pharmaceutical composition containing the agent(s), wherein the pharmaceutical composition comprises a pharmaceutically acceptable carrier vehicle, or the like.

Sterile pharmaceutical formulations are compounded or manufactured according to pharmaceutical-grade sterilization standards (for example, the United States Pharmacopeia Chapters 797, 1072, and 1211; California Business & Professions Code 4127.7; 16 California Code of Regulations 1751, 21 Code of Federal Regulations 211) known to those of skill in the art.

The compositions comprising a first, epigenetically-acting agent, and a second chemotherapeutic agent can be administered simultaneously (i.e., simultaneous administration) and/or sequentially (i.e., sequential administration).

The term “simultaneous administration,” as used herein, means that the agents are administered with a time separation of no more than about 15 minute(s), such as no more than about any of 10, 5, or 1 minutes. When the agents are administered simultaneously, they may be contained in the same composition (e.g., a composition comprising both the epigenetically-acting agent and the second chemotherapeutic agent) or in separate compositions (e.g., the epigenetically-acting agent is contained in one composition and the second chemotherapeutic agent is contained in another composition).

In some variations, the epigenetically-acting agent and the second chemotherapeutic agent are administered sequentially. The term “sequential administration” as used herein means that the epigenetically-acting agent and the second chemotherapeutic agent are administered with a time separation of more than about 15 minutes, such as more than about any of 20, 30, 40, 50, 60 or more minutes. Either the epigenetically-acting agent or the second chemotherapeutic agent may be administered first. The epigenetically-acting agent and the second chemotherapeutic agent are contained in separate compositions, which may be contained in the same or different packages.

In some variations, the administration of the epigenetically-acting agent and the second chemotherapeutic agent are concurrent, i.e., the administration period of the epigenetically-acting agent and the second chemotherapeutic agent overlap with each other. In some variations, the administration of the epigenetically-acting agent and the second chemotherapeutic agent are non-concurrent. For example, in some variations, the administration of epigenetically-acting agent is terminated before the second chemotherapeutic agent is administered. In some variations, the administration of the second chemotherapeutic agent is terminated before the epigenetically-acting agent is administered. The time period between these two non-concurrent administrations can range from about two to eight weeks, such as about four weeks.

The dosing frequency of the epigenetically-acting agent and the second chemotherapeutic agent may be adjusted over the course of the treatment, based on the judgment of the administering physician. When administered separately, the epigenetically-acting agent and the second chemotherapeutic agent can be administered at different dosing frequency or intervals.

The epigenetically-acting agent and the second chemotherapeutic agent can be administered using the same route of administration or different routes of administration.

The doses required for the epigenetically-acting agent and/or the second chemotherapeutic agent may (but not necessarily) be lower than what is normally required when each agent is administered alone. Thus, in some variations, a subtherapeutic amount of the epigenetically-acting agent and/or the second chemotherapeutic agent are administered. “Subtherapeutic amount” or “subtherapeutic level” refer to an amount that is less than the therapeutic amount, that is, less than the amount normally used when the epigenetically-acting agent and/or the second chemotherapeutic agent are administered alone. The reduction may be reflected in terms of the amount administered at a given administration and/or the amount administered over a given period of time (reduced frequency).

In some variations, enough of the second chemotherapeutic agent is administered so as to allow reduction of the normal dose of the epigenetically-acting agent required to effect the same degree of treatment by at least about any of 5%, 10%, 20%, 30%, 50%, 60%, 70%, 80%, 90%, or more. In some variations, enough of the epigenetically-acting agent is administered so as to allow reduction of the normal dose of the second chemotherapeutic agent required to affect the same degree of treatment by at least about any of 5%, 10%, 20%, 30%, 50%, 60%, 70%, 80%, 90%, or more.

In some variations, the dose of both the epigenetically-acting agent and the second chemotherapeutic agent are reduced as compared to the corresponding normal dose of each when administered alone. In some variations, both the epigenetically-acting agent and the second chemotherapeutic agent are administered at a subtherapeutic, i.e., reduced, level. In some variations, the dose of the epigenetically-acting agent and/or the second chemotherapeutic agent is substantially less than the established maximum toxic dose (MTD). For example, the dose of the epigenetically-acting agent and/or the second chemotherapeutic agent is less than about 50%, 40%, 30%, 20%, or 10% of the MTD.

Kits

The invention also provides kits for use in the instant methods. Kits of the invention comprise one or more containers comprising an epigenetically-acting agent (or unit dosage forms and/or articles of manufacture) and/or a second chemotherapeutic agent, and in some variations, further comprise instructions for use in accordance with any of the methods described herein. The kit may further comprise a description of selection an individual suitable or treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

In some variations, the kit comprises a) a composition comprising an epigenetically-acting agent, b) an effective amount of at least one other chemotherapeutic agent, and c) instructions for administering the epigenetically active agent and the chemotherapeutic agents simultaneously and/or sequentially, for treatment of cancer or blood disorders.

The epigenetically-acting agent and the second chemotherapeutic agent can be present in separate containers or in a single container. It is understood that the kit may comprise one distinct composition or two or more compositions wherein one composition comprises an epigenetically-acting agent and one composition comprises a chemotherapeutic agent.

The kits of the invention are in suitable packaging. Suitable packaging include, but is not limited to, vials, bottles, jars, flexible packaging (e.g., sealed Mylar or plastic bags), and the like. Kits may optionally provide additional components such as buffers and interpretative information.

The instructions relating to the use of the epigenetically-acting agents generally include information as to dosage, dosing schedule, and route of administration for the intended treatment. The containers may be unit doses, bulk packages (e.g., multi-dose packages) or sub-unit doses. For example, kits may be provided that contain sufficient dosages of the epigenetically-acting agent as disclosed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the epigenetically-acting agent and pharmaceutical compositions and instructions for use and packaged in quantities sufficient for storage and use in pharmacies, for example, hospital pharmacies and compounding pharmacies.

### EXAMPLES

### Example 1

### Experimental Methods

Cell Lines, Culture Conditions, and Chemicals

The human lung, anaplastic carcinoma cell line, Calu-6 (ATCC, Manassas, Va.), was maintained in RPMI 1640 media containing 9% fetal bovine serum, and penicillin and streptomycin, at 37° C., 5% CO₂. Compound PG-11047 (N,N′-bis(3-ethylaminopropyl)-cis-but-2-ene-1,4-diamine tetrahydrochloride) was synthesized as previously described (Reddy V K et al., J. Med. Chem. (1998) 41:4723-4732; U.S. Pat. No. 5,889,061). Compound PG-11048 (N,N′-bis(3-ethylaminopropyl)-trans-but-2-ene-1,4- diamine tetrahydrochloride) was synthesized using a similar procedure to 11047, using 1,4-dibromo-trans-2-butene or 1,4-dichloro-trans-2-butene in place of the cis-2-butene-1,4-diester used for 11047. The DNA methyltransferase inhibitor, 5-azacytidine, was purchased from Sigma (St. Louis, Mo.), and the HDAC inhibitor, MS-275, from Alexis Biochemicals (Plymouth Meeting, Pa.). Custom primers for PCR were synthesized by Invitrogen (Carlsbad, Calif.).

Analysis of Global Alterations in Histone Marks Following PG-11047 Exposure.

Calu-6 cells were seeded at 7×10⁵ cells per 25 cm² flask in 5 mL of media and allowed to attach overnight. At the appropriate time, flasks were aspirated, refreshed with media containing PG-11047 at concentrations of 0, 1, 5, or 10 μM, and incubated at 37° C. for 24 or 48 hours. For analysis of specific histone modifications, nuclear protein was harvested using NE-PER Nuclear and Cytoplasmic Extraction Reagents according to the manufacturer's protocol (Pierce Biotechnology, Rockford, Ill.), and Western blotting was performed using modification-specific antibodies to histone H4. Protein was first quantified using the BioRad DC assay (Hercules, Calif.) with absorbance measured at 750 nm. Absorbance was converted to protein concentration using interpolation on a bovine serum albumin standard curve. Nuclear proteins (30 μg per lane) were separated on pre-cast 10% Bis-Tris Novex gels with 1× MES running buffer (Invitrogen), and transferred onto Immun-Blot PVDF membrane (BioRad). Blots were blocked overnight at 4° C. in Odyssey blocking buffer (LI-COR, Lincoln, Nebr.), and proteins of interest visualized using an antibody specific to the substrate of LSD1, H3K4me2, as well as acetylated H3K9 (AcH3K9) (Millipore, Billerica, Mass.), another histone modification associated with active chromatin. Proliferating Cell Nuclear Antigen (PCNA) (Calbiochem, La Jolla, Calif.) was also used and served as a loading control for normalization. Following washes, blots were incubated with species-specific, fluorophore-conjugated secondary antibodies, to allow visualization and quantification of immunoreactive proteins using the Odyssey infrared detection system and software (LI-COR).

RNA Extraction and Gene Expression

For gene re-expression studies using RT-PCR, cells were seeded and treated with PG-11047 in parallel with those described above for nuclear protein. Additional flasks were co-treated with combinations of PG-11047 or 11048 (1, 5, or 10 μM), and either the DNMT1 inhibitor, 5-azacytidine, or the HDAC inhibitor, MS-275. Concentrations of 5-azacytidine utilized were 0.1, 0.5, and 1 μM, and MS-275 was administered at 0.5 and 1 μM. Following incubation (24 or 48 h), total RNA was extracted using TRIzol reagent (Invitrogen) according to the provided protocol. RNA was quantified, and cDNA was synthesized using SuperScript III First Strand Synthesis System (Invitrogen) with oligo-d(T)₂₀ as the primer. SYBR green-mediated, real-time PCR was performed using primer pairs as follows: CDH-13, 5′-GGA CCG AGA GAC TCT GGA AAA TC-3′ (sense) and 5′-GGG TCA TCC TTA TCT TCA ACT GTC-3′ (antisense); p16, 5′-CGG AGG CCG ATC CAG GTC ATG-3′ (sense) and 5′-CAA TCG GGG ATG TCT GAG GGA C-3′ (antisense); sFRP2, 5′-AAG CCT GCA AAA ATA AAA ATG ATG-3′ (sense) and 5′-TGT AAA TGG TCT TGC TCT TGG TCT-3′ (antisense); GATA-4,5′-GGC CGC CCG ACA CCC CAA TCT-3′ (sense) and 5′-ATA GTG ACC CGT CCC ATC TCG-3′ (antisense); and GapDH, 5′-GAA GAT GGT GAT GGG ATT TC-3′ (sense) and 5′-GAA GGT GAA GGT CGG AGT C-3′ (antisense). A total of 40 cycles of amplification were performed on a BioRad MyiQ real-time PCR detection system, with data collection facilitated by the MyiQ software. For each of the qPCR experiments, samples were analyzed in triplicate, and normalized to the GapDH housekeeping gene as an internal control.

### Example 2

Global epigenetic histone modification changes in Calu-6 cells following treatment with PG-11047.

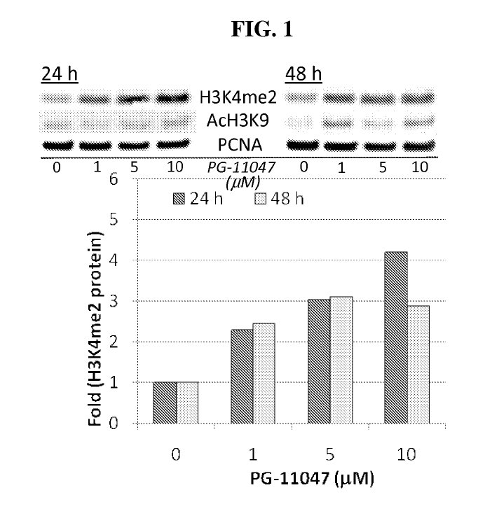

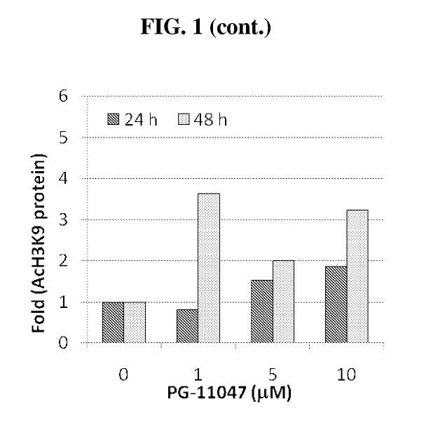

To determine if compound PG-11047 is capable of chromatin modification in the Calu-6 lung carcinoma cell line, cells were treated with increasing doses of the compound for 24 and 48 hours. This exposure resulted in a modest 2-4 fold increase in H3K4me2 protein following 24 hours of treatment. (FIG. 1). Increasing treatment time to 48 hours had no additional effect. Treatment with PG-11047 also increased global amounts of acetylated H3K9 protein 2-3 fold, further indicating the modification of chromatin architecture into a transcriptionally active state.

These data vary somewhat from the high induction of global H3K4me2 seen with the previously reported and validated polyamine analogue-based, LSD1 inhibitors, 2d (N,N″-bis(3,3-diphenylpropyl)-3,21-diimino-2,4,8,16,20,22-hexaazatricosanediimidamide)and PG-11144 ((22E)-N,N′-diethyl-5,10,15,20,25,30,35,40-octaazatetratetracont-22-ene-1,44-diamine). In all cell lines thus far tested, including Calu-6, 2d and PG-11144 demonstrate dramatically increased H3K4me2 levels (10-20 fold), which are induced in a time- and dose-dependent manner.

### Example 3

Increased expression of aberrantly silenced or repressed tumor suppressor genes by PG-11047.

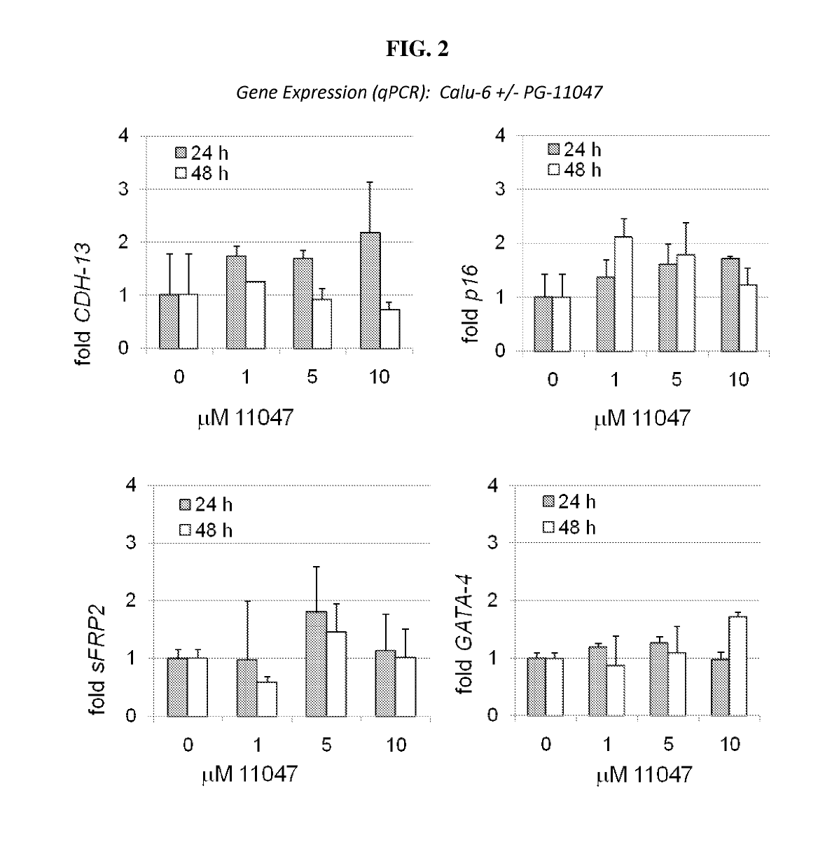

Since H3K4me2 is frequently depleted in the promoter regions of transcriptionally inactive genes, an evaluation was performed to determine if the increase in global H3K4 methylation observed following PG-11047 exposure correlated with increased expression of epigenetically silenced or repressed genes in the Calu-6 cell line. The CDH-13 (h-cadherin), p16, sFRP2, and GATA-4 genes frequently undergo aberrant silencing or repression by CpG island hypermethylation in many types of cancers, both of solid and hematologic origin. RT-PCR analysis of Calu-6 RNA detected very low levels of each of these gene transcripts in untreated Calu-6 cells. Treatment of these cells with PG-11047 appeared to result in an approximate doubling in expression of CDH-13, p16, and sFRP2 mRNA after 24 hours of exposure (FIG. 2) as determined by real-time PCR. GATA-4 mRNA expression levels also appeared to double, but not until 48 hours of treatment.

Combining PG-11047 with other epigenetic-targeting drugs induces additive and synergistic increases in expression of repressed tumor suppressor genes.

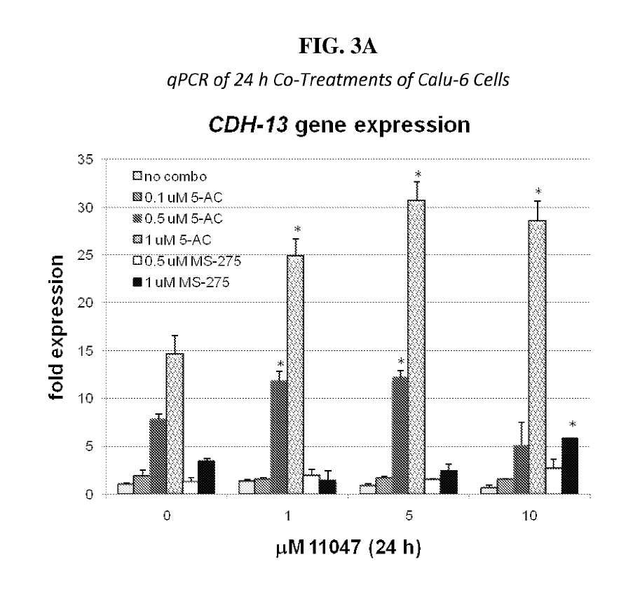

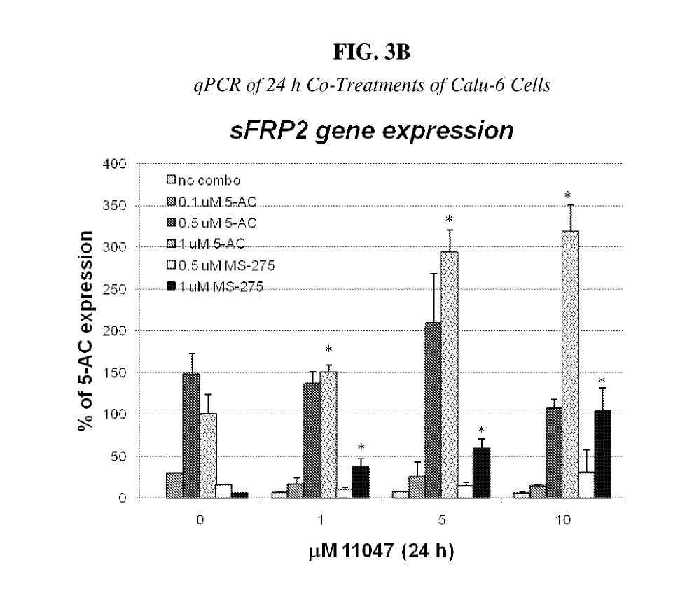

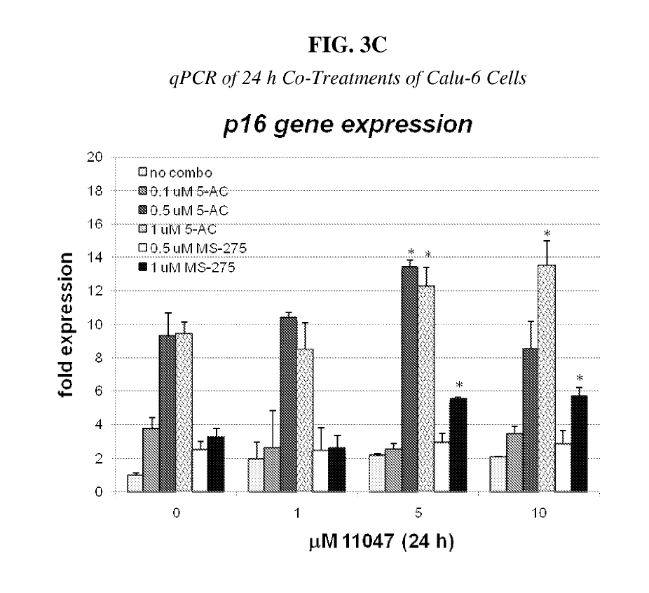

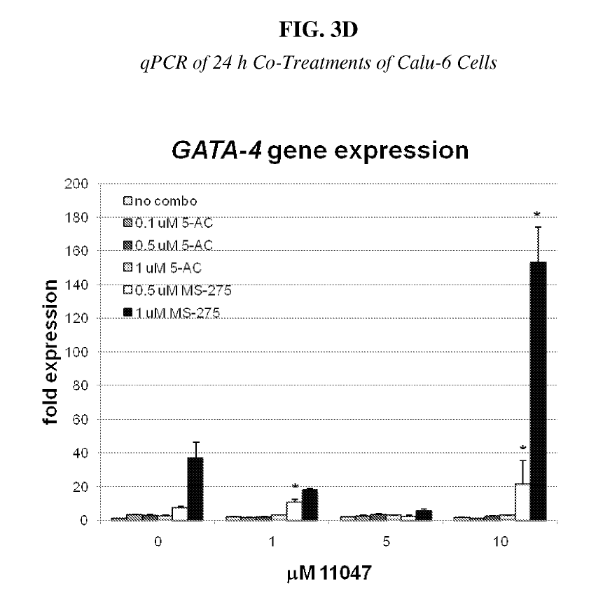

An evaluation was performed to determine if combining the chromatin modifying effects of PG-11047 with those of either DNMT or HDAC inhibitors could induce greater expression of the repressed tumor suppressor genes mentioned above. The Calu-6 cell line was treated with increasing doses of PG-11047, 5-azacytidine, and MS-275, both alone, and in combination, and gene expression was evaluated by real-time PCR after 24 hours (FIG. 3). Co-treatment with PG-11047 and 5-azacytidine produced dramatic effects, with synergistic increases in CDH-13 and sFRP2 gene expression (FIGS. 3A and B). The combination also had at least additive effects on expression of p16 (FIG. 3C). The MS-275 and PG-11047 combination produced additive results for CDH-13, sFRP2, and p16, however the expression level was much lower than those seen with the DNMT inhibitor combinations. Expression of the GATA-4 gene was interesting in that the combination of PG-11047 and 5-azacytidine had no effect, while combining the polyamine with MS-275 gave a synergistic effect and increased gene expression dramatically (FIG. 3D). This is consistent with responses observed when treating with the inhibitors individually, where CDH-13, sFRP2, and p16 gene repression appears to be a result of DNA hypermethylation, while GATA-4 repression can be relieved by treatment with HDAC inhibitor alone, and is presumably dependent upon histone acetylation.

### Example 4

Combining PG-11048 with other epigenetic-targeting drugs induces additive and synergistic increases in expression of repressed tumor suppressor genes.

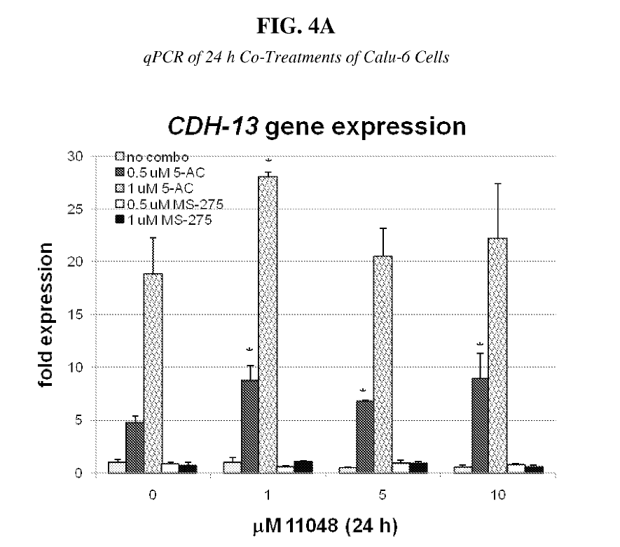

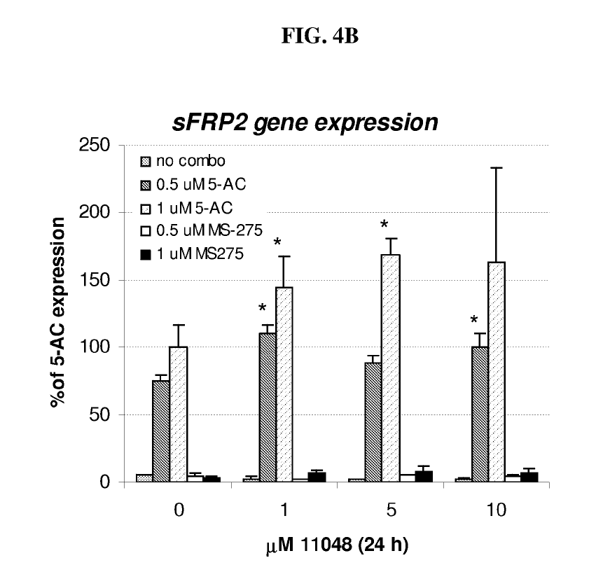

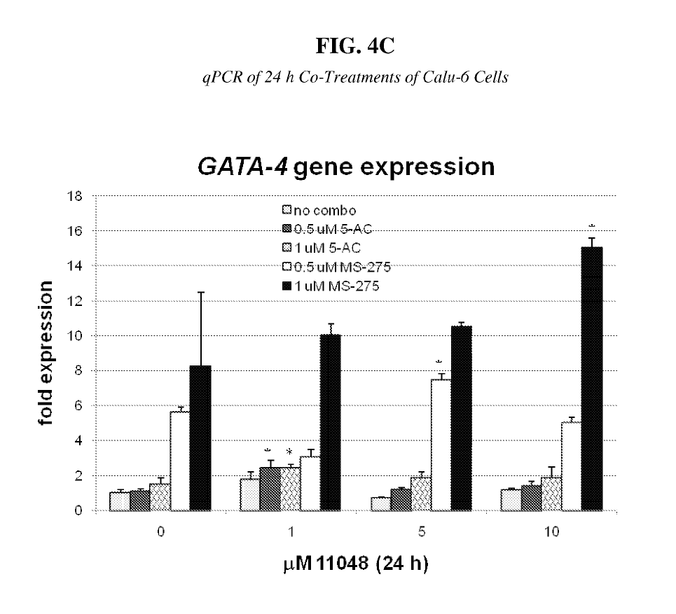

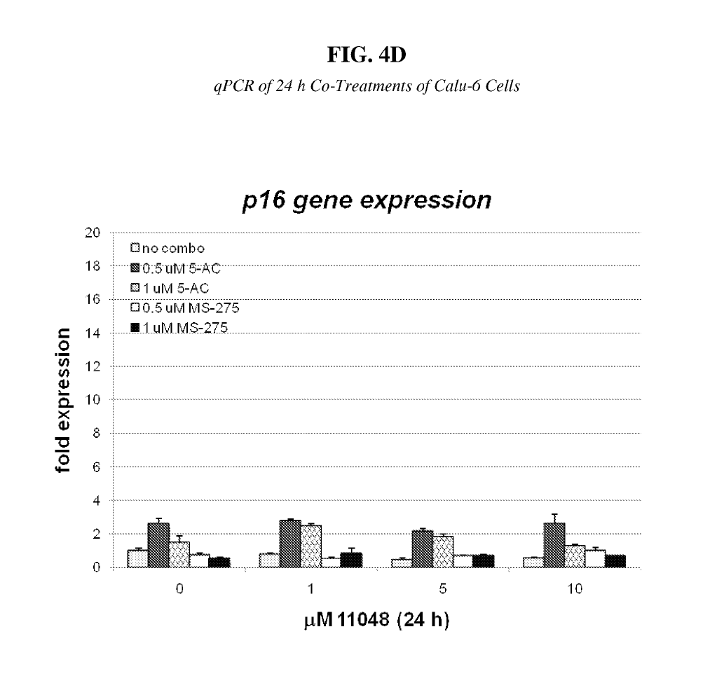

Cells were assayed to determine if combining PG-11048 with either DNMT or HDAC inhibitors could induce greater expression of the repressed tumor suppressor genes mentioned above. The Calu-6 cell line was treated with increasing doses of PG-11048, 5-azacytidine, and MS-275, both alone, and in combination, and gene expression was evaluated by real-time PCR after 24 hours (FIG. 4). Co-treatment with PG-11048 and 5-azacytidine produced at least additive increases in CDH-13, sFRP2, and possibly GATA-4 gene expression (FIG. 4A-C). The MS-275 and PG-11048 combination produced additive results for sFRP2, and was synergistic in regard to GATA-4 gene expression (FIG. 4B, FIG. 4C). Neither of the combinations had any effect on expression of p16 (FIG. 4D).

### Example 5

### PG-11047 in Combination With 5-Azacytidine In Vivo

MC7 cells were injected into mouse flanks and allowed to establish. Mice were dosed with 5-azacytidine, 0.5 mg/kg per day, on days 1-5, 8-12, and 15-19 of a 28-day dosing cycle. PG-11047 (100 mg/kg) was administered on days 2, 9, and 16. No drugs were administered on days 6, 7, 13, 14, or 20-28 of the 28-day cycle. At the end of the 28-day cycle, another cycle of treatment was started. Drugs were administered intraperitoneally. Tumor volume was measured.

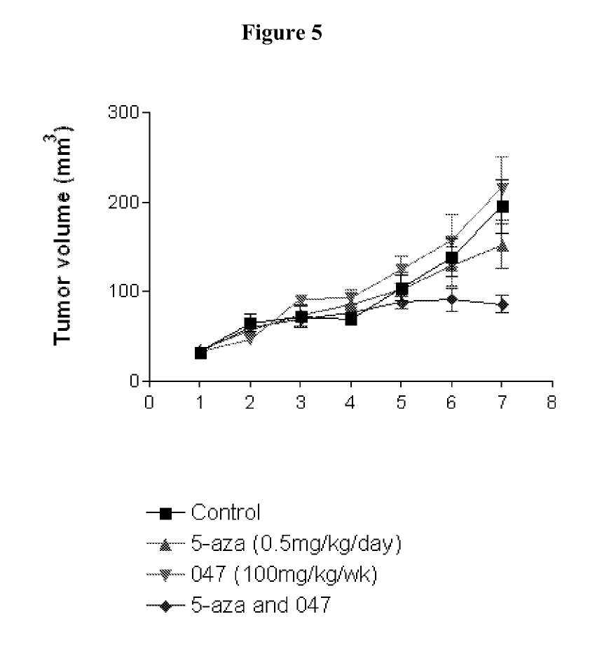

FIG. 5 shows the results of treatment with control, 5-azacytidine (0.5 mg/kg/day) alone, PG-11047 (100 mg/kg/week) alone, and with the combination of 5-azacytidine and PG-11047. At week 7, the tumor volume in the mice receiving the drug combination was substantially lower than the tumor volume in the mice receiving control, 5-azacytidine alone, or PG-11047 alone.

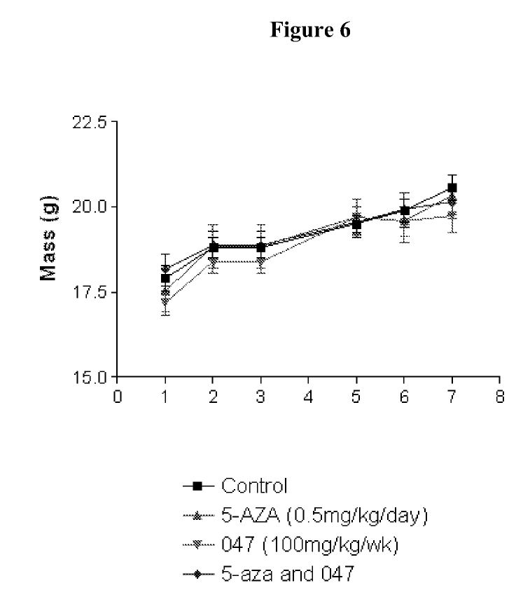

FIG. 6 shows the mass of the animals during the course of the experiment, and shows that the combination of PG-11047 and 5-azacytidine did not affect normal weight gain.

### Example 6

### PG-11047 in Combination With 5-Azacytidine on Tumor Cells Treated Ex Vivo

MCF7 cells were treated in culture for 3 days with 500 nM 5-azacytidine. On the fourth day they were exposed to 5 μM PG-11047 for 24 hours. The treated cells were then implanted (10⁶ cells) into NOD/SCID mice and tumor volumes measured weekly. (The mice were also implanted with slow release estradiol pellets (50 μg), replaced every 6 weeks, as MCF7 cells are estrogen dependent.)

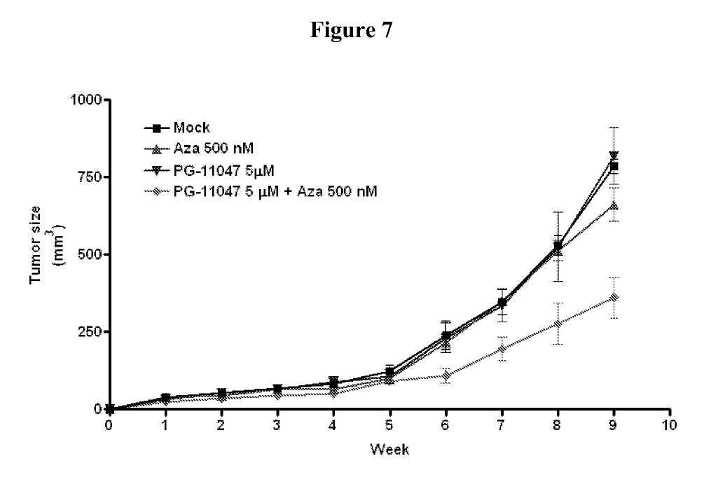

FIG. 7 shows the tumor volume measured in the mice. Treatment of cells with 5-azacytidine and PG-11047 resulted in substantially smaller tumors at nine weeks post-implantation, as compared to cells treated with 5-azacytidine alone, PG-11047 alone, or control.

The disclosures of all publications, patents, patent applications and published patent applications referred to herein by an identifying citation are hereby incorporated herein by reference in their entireties.

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

What is claimed is:

1. A pharmaceutically acceptable composition comprising a combination of PG-11047 ((2Z)-N1,N4-bis [3 -(ethylamino)propyl]-2-butene-1,4-diamine) or PG-11048 ((2E)-N1,N4-bis[3-(ethylamino)propyl]-2-butene-1,4-diamine), and one or more epigenetically-acting drugs.

2. The composition of claim 1, wherein the one or more epigenetically-acting drugs comprise a chemotherapeutic agent.

3. The composition of claim 1, wherein the one or more epigenetically-acting drugs comprise a DNA methyltransferase inhibitor.

4. The composition of claim 1, wherein the one or more epigenetically-acting drugs comprise a histone deacetylase (HDAC) inhibitor.

5. The composition of claim 1, wherein the one or more epigenetically-acting drugs comprise a DNA methyltransferase inhibitor and a histone deacetylase (HDAC) inhibitor.

6. The composition of any of claim 1, additionally comprising one or more additional drugs.

7. The composition of claim 6, wherein the one or more additional drugs comprise one or more chemotherapeutic agents.

8. The composition of claim 1, comprising a combination selected from PG-11047 and 5-azacytidine; PG-11047 and decitabine; PG-11047 and Zebularine; PG-11047 and SGI-110; PG-11047 and RG108; PG-11047 and DZNep; PG-11047 and sodium phenylbutyrate; PG-11047 and valproic acid; PG-11047 and vorinostat; PG-11047 and panobinostat; PG-11047 and belinostat; PG-11047 and JNJ-26481585; PG-11047 and romidepsin; PG-11047 and entinostat; PG-11047 and MGCD-0103; PG-11047, vorinostat (SAHA) and 5-azacytidine; PG-11047, vorinostat (SAHA) and decitabine; PG-11047, vorinostat (SAHA) and NPI-0052 (Salinosporamide A); PG-11047, entinostat (MS-275, SNDX-275) and 5-azacytidine; PG-11047, panobinostat (LBH589) and decitabine; PG-11047, valproic acid and 5-azacytidine; PG-11047, valproic acid and decitabine; PG-11047, valproic acid, 5-azacytidine, and ATRA (all-trans retinoic acid); PG-11047, belinostat and 5-azacytidine; or PG-11047, MGCD-0103 and 5-azacytidine.

9. The composition of claim 1, comprising a combination selected from PG-11048 and 5-azacytidine; PG-11048 and decitabine; PG-11048 and Zebularine; PG-11048 and SGI-110; PG-11048 and RG108; PG-11048 and DZNep; PG-11048 and sodium phenylbutyrate; PG-11048 and valproic acid; PG-11048 and vorinostat; PG-11048 and panobinostat; PG-11048 and belinostat; PG-11048 and JNJ-26481585; PG-11048 and romidepsin; PG-11048 and entinostat; PG-11048 and MGCD-0103; PG-11048, vorinostat (SAHA) and 5-azacytidine; PG-11048, vorinostat (SAHA) and decitabine; PG-11048, vorinostat (SAHA) and NPI-0052 (Salinosporamide A); PG-11048, entinostat (MS-275, SNDX-275) and 5-azacytidine; PG-11048, panobinostat (LBH589) and decitabine; PG-11048, valproic acid and 5-azacytidine; PG-11048, valproic acid and decitabine; PG-11048, valproic acid, 5-azacytidine, and ATRA (all-trans retinoic acid); PG-11048, belinostat and 5-azacytidine; or PG-11048, MGCD-0103 and 5-azacytidine.

10. A method of treating cancer or a blood disorder, comprising administering one or more of the compositions of claim 1 to a patient having cancer or a blood disorder.

11. The method of claim 10, wherein the cancer is selected from the group comprising hematopoietic cancers, acute myeloid leukemia, lung cancers, mesothelioma, cutaneous T-cell lymphoma (CTCL), multiple myeloma, and solid tumors.

12. The method of claim 10, wherein the blood disorder is myelodysplastic syndrome.

13. The method of claim 12, wherein the myelodysplastic syndrome is selected from refractory anemia, refractory anemia with ringed sideroblasts, refractory anemia with excess blasts, refractory anemia with excess blasts in transformation, refractory cytopenia with multilineage dysplasia, myelodysplastic syndrome associated with an isolated del(5q) chromosome abnormality, or unclassifiable myelodysplastic syndrome.

14. A method of treating cancer or a blood disorder in an individual, comprising administering to the individual: a) an effective amount of a composition comprising an epigenetically-acting drug, and b) an effective amount of PG-11047 or PG- 11048.

15. The method of claim 14, wherein the individual is a human.
